(19) United States
(12) Patent Application Publication
(10) Pub. No.: US 2006/0287284 A1
(43) Pub. Date: Dec. 21, 2006

(54) Pharmaceutical composition combining tenatoprazole and an anti-inflamatory agent

(75) Inventors: Francois Schutze, Saint-Nom-La-Breteche (FR); Suzy Charbit, Créteil (FR); Hervé Ficheux, Nogent-Sur-Marne (FR); Michel Homerin, Courcouronnes (FR); Alain Taccoen, Le Chesnay (FR); Nathalie Taccoen, Le Chesnay (FR); Yoshio Inaba, Chuo-Ku, Tokyo (JP)

Correspondence Address: BUCHANAN, INGERSOLL & ROONEY PC, POST OFFICE BOX 1404, ALEXANDRIA, VA, 22313-1404, US

(73) Assignees: Negma Gild, Toussus Le Noble (FR); Mitsubishi Pharma Corporation, Tokyo (JP)

(21) Appl. No.: 10/532,041

(22) PCT Filed: Oct. 21, 2003
(86) PCT No.: PCT/FR03/03120

§ 371 Date: Jun. 23, 2006

(30) Foreign Application Priority Data
Oct. 21, 2002 (FR) 02/13115

(51) Int. Cl. A61K 31/4439; A61K 31/192; A61K 31/60

(52) U.S. Cl. 514/165; 514/338; 514/570; 514/569

(57) ABSTRACT

The invention relates to a novel pharmaceutical combination. The inventive pharmaceutical composition comprises a combination of tenatoprazole and one or more anti-inflammatory agents preferably selected from non-steroid anti-inflammatory agents and cyclooxygenase-2 inhibitors. The invention is suitable for the treatment of painful and inflammatory manifestations.

The present invention concerns a new composition for therapeutic purposes, and more particularly a new pharmaceutical composition combining an anti-inflammatory and tenatoprazole to treat the symptoms of pain and inflammatory diseases while avoiding the adverse effects of standard anti-inflammatory agents.

Anti-inflammatory agents are a class of medicinal products which have been widely employed for many years. One of the first anti-inflammatories used therapeutically was aspirin, the antipyretic, analgesic and platelet anti-aggregant properties of which are also well-recognised and justify its administration in numerous indications. Thus, it is estimated that several millions of aspirin tablets are consumed each year throughout the world.

Non-steroidal anti-inflammatory drugs (NSAIDs) are the medicinal products most widely used to treat pain and acute inflammation. They can mainly be broken down between standard NSAID and cyclo-oxygenase isoenzyme-2 (COX-2) inhibitors.

For example, aspirin, diclofenac, etodolac, indomethacin, naproxen, ibuprofen and piroxicam are standard NSAIDs frequently prescribed to treat the symptoms of inflammatory rheumatism and arthrosis.

However, standard NSAIDs cause certain adverse effects, and in particular tend to induce gastric or intestinal ulcers (Goodman and Gilman, The Pharmacological Basis of Therapeutics; 9th Edition, McGraw Hill). These adverse effects are linked to inhibition of the cyclo-oxygenase-1 enzyme (COX-1), the constitutive isoform. They are particularly problematic because the drug needs to be administered over a long period of time, especially in the treatment of chronic disorders.

Discovery of the existence of another isoform of the cyclo-oxygenase enzyme, cyclo-oxygenase-2 (COX-2), the isoform induced when inflammation occurs, has made it possible to envisage the development of potentially more specific and safer medicinal products. These NSAIDs, which are available today, have the effect of selectively inhibiting the action of COX-2, and thus act on inflammation with a lower incidence of adverse effects in the upper gastrointestinal tract. Thus COX-2 inhibitors such as celecoxib and rofecoxib have been developed, and constitute a new class of drugs to treat the symptoms of inflammatory diseases.

Nevertheless, although COX-2 inhibitors can markedly reduce major disorders, such as gastric or haemorrhagic ulcers linked to the administration of standard NSAIDs, they do not enable a significant improvement in minor problems such as gastric pain and dyspepsia, and do not prevent all major disorders. Thus a recent study showed that the percentages of minor disorders observed in patients receiving celecoxib were 4.8% (gastric pain), 4.8% (dyspeptic symptoms) and 2.4% (nausea), while in the case of treatment with standard NSAIDs, these percentages were 6.2%, 5.9% and 3.4%, respectively. Similar results were obtained when comparing treatment with another COX-2 inhibitor, rofecoxib, with standard NSAIDs.

Tenatoprazole, or 5-methoxy-2-[[(4-methoxy-3,5-dimethyl-2-pyridyl)methyl]sulfinyl]imidazo[4,5-b]pyridine, is described in Patent No. EP 254.588. It belongs to the group of medicinal products considered as proton pump inhibitors, which are useful in the treatment of gastric ulcers.

The first known derivative of this series was omeprazole, described in Patent No. EP 005.129, and endowed with properties which inhibit the secretion of gastric acid; it is widely employed as an anti-ulcerative in human therapeutics.

Other proton pump inhibitors include rabeprazole, pantoprazole and lansoprazole, which all exhibit structural analogy and belong to the group of pyridinyl-methyl-sulfinyl-benzimidazoles. Tenatoprazole has a similar structure, but of the imidazopyridine type. These compounds are sulfoxides presenting with asymmetry at the level of the sulphur atom, and therefore generally take the form of a racemic mixture of two enantiomers.

Omeprazole has also been envisaged for the treatment of gastroesophageal reflux disorders, but its action in this indication is not entirely satisfactory. Thus studies have shown that its duration of action, like that of other proton pump inhibitors, is insufficient to ensure the efficient treatment of nocturnal reflux.

Tenatoprazole is described in detail in Patent No. EP 254.588, together with its ability to inhibit ATPase (H⁺+K⁺) and the secretion of gastric acid.

The prescription of proton pump inhibitors such as omeprazole has already been proposed for patients treated with anti-inflammatories, so as to limit their adverse effects and particularly the complications linked to gastric lesions and ulcers, but the adverse effects of anti-inflammatories can be very severe and unpredictable, particularly in high-risk subjects such as the elderly, and the concomitant administration of a standard proton pump inhibitor does not fully meet the need for preventive therapy.

Patent application WO 01.66088 relates to an autoemulsifying pharmaceutical form for oral administration of a a NO group-releasing NSAID, which forms an emulsion in situ upon contact with the gastric fluids. The possible combination of such an anti-inflammatory agent with a usual proton pump inhibitor such as omeprazole, is also considered. Patent application WO 01.56573 discloses anti-inflammatory agents of the COX2-inhibitors series which are likely to increase the gastro-intestinal motility, and this patent also considers the possible combination with proton pump inhibitors. However the two above cited patent applications do not describe any example of such a combination and they do not suggest to combine an anti-inflammatory agent wpecifically with tenatoprazole.

The combination of an E1 prostaglandin analogue such as misoprostol with an anti-inflammatory such as diclofenac has also been proposed to treat gastric ulcers arising as an adverse effect of an anti-inflammatory, but the elimination half-life of misoprostol is too short to procure a long-term effect.

There thus remains a need for a medicinal product endowed with anti-inflammatory activity which can be used for prolonged courses of treatment without causing harmful effects, particularly in elderly patients or those presenting with risks of gastric or duodenal ulcer, and which will, on the contrary, enable the prevention of such adverse effects.

The aim of the present invention is indeed to make available to practitioners a medicinal product intended for the treatment of painful and inflammatory symptoms, and notably to treat the symptoms of inflammatory diseases such as inflammatory rheumatism, arthritis and osteoarthritis, by exerting a preventive effect against adverse effects causing gastro-duodenal lesions and peptic ulcers.

Studies performed by the applicant have shown that the combination of tenatoprazole and an anti-inflammatory achieves unexpected effects when compared with other proton pump inhibitors and with anti-inflammatories, notably NSAIDs, used alone or in combination. More specifically, it has been shown that the combination of tenatoprazole and one or more anti-inflammatory drugs enables the control of gastric acidity, combined with the anti-inflammatory activity which enables improved efficacy and better safety of use, and allows the effective treatment of patients suffering from pain and inflammatory diseases, particularly rheumatic inflammations such as arthritis, rheumatoid arthritis and arthrosis, while preventing the digestive disorders induced by anti-inflammatory agents.

The object of the present invention is therefore a pharmaceutical composition combining a specific proton pump inhibitor, tenatoprazole, with one or more anti-inflammatory drugs.

The present invention also aims to produce a pharmaceutical preparation for administration via the oral or parenteral routes, comprising tenatoprazole and one or more anti-inflammatory drugs, in a form appropriate to treating the symptoms of painful and inflammatory disorders.

A further object of the present invention is the combined use of tenatoprazole and at least one anti-inflammatory drug to treat painful and inflammatory symptoms, and the combined use of tenatoprazole and at least one anti-inflammatory drug to manufacture a medicinal product aimed at treating the symptoms of painful and inflammatory disorders.

According to the invention, tenatoprazole can be used in a free form or in the form of a salt; for example, a potassium, magnesium, sodium or calcium salt.

The anti-inflammatory agent used in compositions according to the present invention may be chosen from amongst standard non-steroidal anti-inflammatory drugs (NSAIDs) and cyclo-oxygenase-2 inhibitors. Thus it may be possible to combine tenatoprazole and aspirin or a standard NSAID selected from diclofenac, etodolac, indomethacin, naproxen, ibuprofen or piroxicam. The cyclo-oxygenase-2 inhibitor employed in compositions according to the invention could, for example, be celecoxib or rofecoxib.

Compositions according to the present invention may be used advantageously, as shown above, for any treatment of painful and inflammatory symptoms, particularly in elderly patients, those presenting with a history of ulcers, or those receiving treatment with aspirin or anticoagulants, etc. They are particularly suitable for the treatment of inflammatory rheumatisms, notably arthritis andosteoarthritis, painful gums, etc., where they will avoid the major and minor digestive complications linked to the use of known anti-inflammatory agents.

Studies performed by the applicant have shown that these symptoms can be treated effectively with a composition complying with the present invention, combining tenatoprazole and an anti-inflammatory agent, and that the advantage ensured by a lower risk of adverse effects, notably gastro-duodenal lesions and peptic ulcers, results from a specific form of tenatoprazole activity which complements that of the anti-inflammatory drug.

Indeed, tenatoprazole can be distinguished from other proton pump inhibitors by its astonishingly longer elimination half-life, and also its considerable degree of tissue exposure, as has been demonstrated during experiments conducted by the claimant.

Thus, the phase I study in Caucasian individuals (n=8 per group) made it possible to demonstrate the influence of different doses of tenatoprazole on pharmacokinetic parameters, in the case of the oral administration of a single dose and a daily dose for a period of 7 days.

The doses tested were 10, 20, 40 and 80 mg of tenatoprazole.

The results obtained are grouped in Table 1 below.

TABLE 1

| | Single dose | | | | Repeated doses (7 days) | | | |
|---|---|---|---|---|---|---|---|---|
| | 10 mg | 20 mg | 40 mg | 80 mg | 10 mg | 20 mg | 40 mg | 80 mg |
| Cmax (μg/ml) | 0.9 | 2.4 | 5.3 | 8.3 | 1.6 | 3 | 5.5 | 11.8 |
| Tmax (h) | 4 | 4 | 3 | 3 | 3 | 2 | 3 | 2 |
| T½ (h) | 5 | 6 | 6 | 7 | 5 | 8 | 9 | 9.2 |
| AUC 0-t | 8 | 24 | 43 | 97 | 13 | 36 | 75 | 218 |

In this table, the abbreviations employed have the following meanings:

Cmax maximum concentration

Tmax time required to attain maximum concentration

T1/2 elimination half-life

AUC₀₋ₜ area under the curve, between time 0 and the last measurable concentration.

The results shown in Table 1 above demonstrate that the mean elimination half-lives were between 5 and 6 hours after the administration of a single dose, and between 5 and 9.5 hours after administration for seven days, depending on the dose. Tenatoprazole also exhibited high AUC values (area under the curve), providing evidence of a low rate of metabolism and/or high bioavailability via the oral route. Furthermore, whatever the conditions of administration, single or repeated, the Cmax, AUC₀₋ₜ and AUC_{0-inf} values increased in a linear fashion. The AUC_{0-inf} value was calculated by extrapolation.

A comparison of AUC values between two proton pump inhibitors, lansoprazole and omeprazole, had already been made by Tolman et al. (J. Clin. Gastroenterol., 24(2), 65-70, 1997), but this did not enable a judgement as to the superiority of one product over the other. Indeed, different criteria must be taken into account, i.e. the time required for pump regeneration, the period above the minimum concentration necessary to inhibit proton pumps. With respect to the pump regeneration time, it is observed that pumps usually have a half-life of about 30 to 48 hours, and are therefore totally renewed every 72 to 96 hours.

The pharmacokinetic study performed by the applicant showed that, thanks to the unexpected pharmacokinetic properties described above, tenatoprazole could counteract the proton pump regeneration phenomenon by maintaining an inhibitory concentration for a sufficiently long period of time to meet the two criteria specified previously.

Thus, the prolonged exposure linked to the long elimination half-life of tenatoprazole, and demonstrated by the AUC value, endows it with longer presence at the sites of activity and thus procures a pharmacodynamic effect which is prolonged over time. Experiments have thus shown that tenatoprazole is endowed with a plasma half-life/pump regeneration time ratio which is notably higher than that seen with other proton pump inhibitors, thus permitting its use in pathologies where currently available medicinal products have little effect, and particularly treatment of the nocturnal symptoms of gastroesophageal reflux and gastro-duodenal ulcers.

Therefore, when it is combined with an anti-inflammatory, such as diclofenac, celecoxib, indomethacin, naproxen, ibuprofen or rofecoxib, and preferably administered in the evening before going to bed, tenatoprazole, when compared with other proton pump inhibitors, procures a significant advantage with respect to suppressing gastric acidity, and consequently allows effective action on the nocturnal peak of gastric acidity and on nocturnal symptoms in patients suffering from gastroesophageal reflux, in which it achieves marked relief, even in patients refractory to classic therapies with standard proton pump inhibitors such as omeprazole.

The composition of the present invention can be administered in standard forms adapted to the method of administration chosen, for example via the oral or parenteral routes, and preferably via the oral or intravenous routes. For example, it is possible to use formulations of tablets or capsules containing tenatoprazole and the anti-inflammatory as the active substances, or emulsions or solutions for parenteral administration containing a tenatoprazole salt combined with one or more anti-inflammatory agents, and a standard, pharmaceutically acceptable substrate.

The unit doses may contain between 10 and 60 mg tenatoprazole and between 10 and 500 mg of the anti-inflammatory agent, particularly diclofenac, naproxen, ibuprofen, celecoxib or rofecoxib.

As an example, an appropriate formulation for a capsule containing tenatoprazole combined with a standard, nonsteroidal anti-inflammatory agent, is given below:

| | | |
|---|---|---|
| Tenatoprazole | 20 | mg |
| Diclofenac | 100 | mg |
| excipients | qs 300 | mg |

An example of a formulation combining tenatoprazole and a cyclo-oxygenase inhibitor is given below:

| | | |
|---|---|---|
| Tenatoprazole | 20 | mg |
| Celecoxib | 200 | mg |
| excipients | qs 300 | mg |

The dosage is determined by the practitioner as a function of the patient's state and severity of the disorder. It is generally between 10 and 120 mg, preferably between 20 and 40 mg, of tenatoprazole per day, for 20 to 1 600 mg of the anti-inflammatory agent.

For example, treatment for a painful, inflammatory episode of osteoarthritis in the knee in an elderly subject could consist in the administration of 1 to 2 tablets, each containing 20 mg tenatoprazole and 100 mg diclofenac, every evening for a period of between 4 and 10 weeks, in the case of initial or maintenance therapy.

In patients with severe disorders, it may be effective to administer the medicinal product via the intravenous route in the first instance, and subsequently via the oral route.

The invention also has the advantage of permitting sequential treatment which is effective using a single dose each week of one tablet containing 20 or 40 mg tenatoprazole combined with 100 to 200 mg of the anti-inflammatory agent, for example diclofenac, celecoxib or rofecoxib.

The study of clinical cases described below demonstrated the efficacy of the combination described in the invention.

TABLE 2
Prevention of digestive disorders

| | NSAID/ | | Duration | Severe | Minor | |
|---|---|---|---|---|---|---|
| Age/ | tenatoprazole | Weight | of | dig. | dig. | |
| gender | combination | ratio | treatment | disorder | disorder | Safety |
| 45/F | Naproxen/T | 500/20 | 8 wks. | 0 | 0 | +++ |
| 39/F | Diclofenac/T | 100/20 | 12 wks. | 0 | 0 | +++ |
| 41/M | Ibuprofen/T | 600/20 | 8 wks. | 0 | 0 | +++ |
| 34/M | Diclofenac/T | 100/20 | 8 wks. | 0 | 0 | +++ |
| 52/M | Celecoxib/T | 200/20 | 8 wks. | 0 | 0 | +++ |
| 39/M | Celecoxib/T | 200/20 | 10 wks. | 0 | 0 | +++ |

T = tenatoprazole

The weight ratio between the NSAID and tenatoprazole is expressed in mg. Thus “Naproxen/T” “500/20” means a capsule combining 500 mg naproxen and 20 mg tenatoprazole. The treatment comprised the administration of one capsule per day during the period mentioned above. In the case of the assocaition of ibuprofen and tenatoprazole, each capsule contained 400 mg of ibuprofen and 5 mg of tenatoprazole, which was administered as 4 capsules per day.

The results reported in Table 2 above show that the administration of a composition according to the invention combining tenatoprazole and a non steroidal anti-inflammatory agent, did not resulted in any heavy or minor digestive trouble, and that the treatment was very well tolerated.

1. Pharmaceutical composition for use in the treatment of pain and inflammatory diseases, wherein it comprises a combination of tenatoprazole and one or more anti-inflammatory agents.

2. Composition according to claim 1, wherein the anti-inflammatory agent is selected from nonsteroidal anti-inflammatory agents and cyclo-oxygenase-2 inhibitors.

3. Composition according to claim 2, wherein the nonsteroidal anti-inflammatory agent is aspirin.

4. Composition according to claim 2, wherein the nonsteroidal anti-inflammatory agent is selected from diclofenc, etodolac, indometacin, naproxen, ibuprofen and piroxicam.

5. Composition according to claim 2, wherein the cyclo-oxygensase-2 inhibitor is selected from rofecoxib and celecoxib.

6. Composition according to claim 1, wherein the weight ratio tenatoprazole:anti-inflammatory agent is comprised between 1:2 and 1:40.

7. Composition according to claim 1, wherein the composition comprises unitary doses each containing from 10 to 60 mg of tenatoprazole and from 10 to 500 mg of anti-inflammatory agent.

8. Composition according to claim 1, wherein tenatoprazole is in the form of a potassium, magnesium, sodium or calcium salt.

9. Composition according to claim 1, wherein the composition is administrable via the oral or the parenteral route.

10. (canceled)

11. Composition according to claim 2, wherein the weight ratio tenatoprazole anti-inflammatory agent is comprised between 1:2 and 1:40.

12. Composition according to claim 3, wherein the weight ratio tenatoprazole anti-inflammatory agent is comprised between 1:2 and 1:40.

13. Composition according to claim 4, wherein the weight ratio tenatoprazole anti-inflammatory agent is comprised between 1:2 and 1:40.

14. Composition according to claim 5, wherein the weight ratio tenatoprazole anti-inflammatory agent is comprised between 1:2 and 1:40.

15. Composition according to claim 2, wherein the composition comprises unitary doses each containing from 10 to 60 mg of tenatoprazole and from 10 to 500 mg of anti-inflammatory agent.

16. Composition according to claim 3, wherein the composition comprises unitary doses each containing from 10 to 60 mg of tenatoprazole and from 10 to 500 mg of anti-inflammatory agent.

17. Composition according to claim 4, wherein the composition comprises unitary doses each containing from 10 to 60 mg of tenatoprazole and from 10 to 500 mg of anti-inflammatory agent.

18. Composition according to claim 5, wherein the composition comprises unitary doses each containing from 10 to 60 mg of tenatoprazole and from 10 to 500 mg of anti-inflammatory agent.

19. Composition according to claim 6, wherein the composition comprises unitary doses each containing from 10 to 60 mg of tenatoprazole and from 10 to 500 mg of anti-inflammatory agent.

20. Composition according to claim 2, wherein the composition is administrable via the oral or the parenteral route.

21. A medicament for the treatment of pain and inflammatory diseases composed of tenatoprazole and one or more anti-inflammatory agents.
